(19) United States
(12) Patent Application Publication
(10) Pub. No.: US 2008/0110082 A1
(43) Pub. Date: May 15, 2008

(54) Biodiesel production with enhanced alkanol recovery

(76) Inventors: Thomas Arthur Maliszewski, Charleston, WV (US); Donald Leroy Bunning, South Charleston, WV (US); Louis A. Kapicak, Cross Lanes, WV (US); David James Schreck, Lake City, MN (US)

Correspondence Address: Nick C. Kottis;Pauley Petersen & Erickson, Suite 365, 2800 W. Higgins Road, Hoffman Estates, IL, 60169, US

(21) Appl. No.: 11/901,967

(22) Filed: Sep. 19, 2007

Related U.S. Application Data
(60) Provisional application No. 60/845,725, filed on Sep. 19, 2006.

(51) Int. Cl. C10L 1/18; B01J 8/00; C07C 29/76

(52) U.S. Cl. 44/388; 422/188; 568/913

(57) ABSTRACT

Processes for making biodiesel are improved by fast, vapor fractionating a crude biodiesel containing alkyl ester, lower alkanol and a catalytically effective amount of base catalyst to obtain a lower alkanol fraction having a low content of water without undue loss of alkyl ester despite the presence of active catalyst.

### CROSS REFERENCE TO RELATED APPLICATIONS

This application claims the benefit of and priority to U.S. Provisional Application No. 60/845,725, filed Sep. 19, 2006, the entirety of which application is incorporated herein by reference.

### FIELD OF THE INVENTION

This invention pertains to processes for the synthesis of biodiesel from fats and oils by base catalyzed transesterification with lower alkanol, and particularly to such processes where unreacted lower alkanol is recovered from crude biodiesel in an economically viable manner and at a purity suitable for recycling to the transesterification.

### BACKGROUND TO THE INVENTION

Biodiesel is being used as an alternative or supplement to petroleum-derived diesel fuel. Biodiesel can be made from various bio-generated oils and fats from vegetable and animal sources.

One process involves the transesterification of triglycerides in the oils or fats with a lower alkanol in the presence of a base catalyst to produce alkyl ester useful as biodiesel and a glycerin co-product. In this process, the alkyl ester and glycerin are separated, usually by a phase separation, and the lighter phase containing crude biodiesel is refined. In typical refining operations, the catalyst is neutralized by the addition of an aqueous acid solution to convert the catalyst to a salt, and then lower alkanol, the salts and any soaps formed by the saponification of free fatty acids during the base-catalyzed transesterification are removed. In this refining procedure, the neutralized crude biodiesel is washed with water to remove salts, lower alkanol and residual glycerin. The washed biodiesel may be dried. The spent wash water is then fractionated to provide a lower alkanol stream suitable for recycle to the transesterification reactor system or is discarded. The water must be removed from the lower alkanol if the lower alkanol is to be recycled to the transesterification reaction zone since water is reactive and can lead to the formation of soaps rather than the sought alkyl esters and can lead to loss of base catalyst through disassociation.

The transesterification is an equilibrium limited reaction. Hence, an excess of lower alkanol would be beneficial for enhancing the production of alkyl ester for biodiesel. However, a balance exists between the desire to use a stoichiometric excess of lower alkanol and the costs associated with the use of such excesses of lower alkanol.

Alasti, in U.S. 2006/0074256 discloses a biodiesel process having a refining system in which a feed containing mono-alkyl esters, glycerol, alcohol and salts is subjected to a separation by volatility to remove alcohol and a subsequent separation by volatility to provide a vapor stream containing mono-alkyl esters and glycerol. A further separation by volatility separates mono-alkyl esters from glycerol. In paragraph 23 various evaporator types are disclosed for the separation of alcohol including forced circulation, rising film and falling film evaporators. A horizontal, thin or wiped rotary blade evaporator is preferred.

Various processes are commercially offered for making biodiesel by transesterification of triglycerides. Desmet Ballestera have a process in which crude biodiesel from transesterification is mixed with a water and citric acid mixture. The admixture is subjected to centrifugation to provide a spent wash water phase that is passed to glycerin purification and an oil fraction that is dried to provide a methanol and water vapor phase. Water is recovered from the vapor phase and recycled for the wash. Kemper, Desmet Ballestra Biodiesel Production Technology, Biodiesel Short Course, Quebec City, Canada, May 12-13, 2007.

Crown Iron Works Company has a process for making biodiesel in which the transesterification product is passed to a reactor/neutralizer to which an acid stream is passed. The effluent from the reactor/neutralizer is decanted and the oil phase is centrifuged to remove water which is recycled to the reactor/neutralizer. The oil phase from the centrifuge is passed to a biodiesel stripper. Methanol is recovered and subjected to rectification and recycled to the transesterification section. Waranica, Crown Iron Works Biodiesel Production Technology, Biodiesel Short Course, Quebec Canada.

Accordingly, biodiesel production processes are sought that are capable of recovering lower alkanol from crude biodiesel in an economically attractive manner with the recovered lower alkanol having suitable purity to be recycled for transesterification thereby minimizing the loss of unreacted lower alkanol.

### SUMMARY OF THE INVENTION

By this invention, processes for making biodiesel are provided that recover lower alkanol from crude biodiesel in an economically attractive manner. In accordance with the invention, lower alkanol is removed by fast, vapor fractionation prior to neutralization of the base catalyst. Neutralization of base catalyst with acid co-produces water which would likely be vaporized with unreacted alkanol in refining the crude biodiesel. Moreover, most available acids for neutralization contain some water. By avoiding a prior neutralization, the processes of this invention provide a crude biodiesel that contains reduced water. Thus the water content in the separated lower alkanol fraction can be sufficiently low that the lower alkanol fraction can be recycled without a further unit operation to remove water. Surprisingly, although the transesterification is an equilibrium reaction, the removal of lower alkanol by fast fractionation can occur with virtually no loss in biodiesel such as to monoglycerides.

In its broad aspects, the processes of this invention comprise subjecting crude biodiesel containing alkyl esters of fatty acids (“alkyl esters”), lower alkanol, and a catalytically effective amount of base catalyst, and optionally glycerin and soaps of fatty acids (“soaps”), wherein the crude biodiesel contains less than about 0.5, sometimes less than about 0.1, preferably less than about 0.05 mass percent water, to fast, vapor fractionation conditions to provide a lower boiling fraction containing lower alkanol. The preferred lower alkanols are methanol, ethanol and isopropanol with methanol being the most preferred.

Fast fractionation means that the residence time of the crude biodiesel for the vapor fractionation is sufficiently short under the conditions of the fractionation that virtually no loss of biodiesel occurs by reason of the change in equilibrium as the lower alkanol is separated. Generally the residence time is less than about one minute, and preferably less than about 30 seconds, and sometimes as little as 5 to 25 seconds. Preferably the vapor fractionation conditions comprise a maximum temperature of less than about 200° C., preferably less than about 150° C. or 140° C., and most preferably, when the lower alkanol is methanol, less than about 120° C., especially where the fractionation is under vacuum conditions. Where the alkanol is methanol, the maximum temperature is in the range of about 60° C. to 120° C., and more preferably in the range of about 80° C. to 110° C. Depending upon the lower alkanol, the lower boiling fractionation may need to be conducted under subatmospheric pressure to maintain desired overhead and maximum temperatures.

To further enhance the separation it may be advantageous to introduce an inert gas such as nitrogen to the fractionation. The presence of an inert gas will enhance the removal of the alkanol from the crude biodiesel for any given pressure and temperature of fractionation. However, the presence of the inert gas will reduce the amount of subsequent condensation of the alkanol, reducing the overall alkanol recovery and perhaps increasing the losses of alkanol to the environment. The designer has to manage temperature, pressure, and amount of inert injected to achieve the optimum conditions.

The fast fractionation may be effected by any suitable vapor fractionation technique including, but not limited to, distillation, stripping, wiped film evaporation, and falling film evaporation. Falling film evaporation is preferred due to the control of the surface temperature, the ability to obtain more than one theoretical distillation plate, and the ability to use upwardly flowing vapor phase to sweep the downwardly flowing liquid. Often the falling film evaporator has a tube length of at least about 1 meter, say, between about 1.5 and 5 meters, and an average tube diameter of between about 2 and 10 centimeters.

It is preferred that at least a portion of glycerin in the crude biodiesel is removed by phase separation prior to the fast, vapor fractionation. Often the glycerin content of the crude biodiesel subjected to the fast, vapor fractionation to remove alkanol is less than about 5, preferably less than about 3, and often less than about 1, mass percent. The glycerin may be separated subsequent to the transesterification or between stages of the transesterification if more than one stage is used or both. As water preferentially is sorbed in the glycerin layer, additional means are provided to maintain a low water content in the crude biodiesel being subjected to the fast, vapor fractionation to recover alkanol of sufficient purity to be recycled for transesterification. Generally the water content of the separated alkanol is less than about 0.1, preferably less than about 0.05, mass percent.

In preferred aspects, the processes also pertain to the base catalyzed transesterification of glycerides with lower alkanol. These processes comprise:
a. contacting a glyceride-containing feed and lower alkanol under transesterification conditions comprising the presence of a base catalyst wherein the molar ratio of lower alkanol to glyceride is at least about 3.15:1, preferably between about 3.6:1 to 15:1, and most preferably between about 4.5:1 to 6:1, to provide a crude biodiesel containing alkyl esters of fatty acids, glycerin, lower alkanol, base catalyst and, optionally, soaps of fatty acids, said contacting being for a time sufficient to convert at least about 90, preferably at least about 95, and most preferably at least about 98, mass percent of the glycerides in the glyceride-containing feed;
b. separating by phase separation said crude biodiesel to provide a heavier glycerin-containing layer and a lighter alkyl ester-containing layer, wherein a portion of the water and a portion of the base catalyst are contained in each of the heavier and lighter layer;
c. subjecting the lighter layer while it contains a catalytically effective amount of base catalyst to fast, vapor fractionation conditions to provide a lower boiling fraction containing lower alkanol and a higher boiling fraction containing alkyl esters and base catalyst;
d. recycling at least a portion of the lower boiling fraction of step (c) to step (a) as a portion of the lower alkanol; and
e. contacting the higher boiling fraction with an aqueous acid solution in an amount sufficient to at least neutralize the base catalyst.

In one preferred aspect, step (a) of the processes for the base-catalyzed transesterification of glycerides comprises at least two sequential stages, or zones, each of which is fed lower alkanol, and between stages, glycerin is separated by phase separation. The term reaction stages is not intended to be defined by the number of vessels. A countercurrent flow reactor may thus have multiple stages, or zones. If desired, a plurality of reactor vessels can be used with each defining a reaction stage. Step (b) may thus be performed by phase separation between stages or by phase separation between stages and after the final stage. Additional lower alkanol and base catalyst may be added, if desired, to the lighter layer passing to a subsequent reaction zone. Not only does this sequential process facilitate reaching a high conversion of glyceride, but also, the intermediate separation removes a portion of the water introduced into the reaction system with the glyceride-containing feed, water that may be formed in making the catalyst if an alkali metal hydroxide is used, and made in the prior reaction zone, e.g., by the reaction of a free fatty acid with base to form a soap.

In one embodiment, at least about 50 mass percent of the glyceride fed to a preceding reactor is reacted in the preceding reactor, a glycerin-containing phase is separated from the transesterification product of the first reaction zone and a glyceride and alkyl ester-containing layer is fed to a subsequent reaction zone for substantial completion of the transesterification. The transesterification product from the subsequent reaction zone may be subjected to another phase separation to recover glycerin. In another embodiment, the preceding reaction zone effects at least about 90, preferably between about 92 to 98, percent of the conversion of the glyceride; a phase separation of a glycerin-containing layer is effected and substantial completion of the conversion of the glyceride is effected in the subsequent reaction zone and the transalkylation product from the subsequent transalkylation zone is subjected to step (c) without an intervening phase separation unit operation. Where more than one transalkylation reaction zone is used, the ratio of alkanol to glyceride may be the same or different in each zone.

In a preferred aspect, the glyceride containing feed contains less than about 0.5, more preferably less than about 0.1, mass percent water based upon the total mass of the glyceride-containing feed. Preferably the lower boiling fraction contains less than about 0.1, and more preferably less than about 0.05, mass percent water. Preferably the fast, vapor fractionation conditions are as set forth above.

Another broad aspect of the invention pertains to processes for making biodiesel comprising:
a. contacting a glyceride-containing feed and lower alkanol under transesterification conditions comprising the presence of a base catalyst, wherein the molar ratio of lower alkanol to glyceride is at least about 3.15:1 to provide an intermediate containing alkyl esters of fatty acids, glycerin, lower alkanol, base catalyst, and less than about 0.5, preferably less than about 0.1, mass percent water, said contacting being for a time sufficient to convert at least about 90 mass percent of the glycerides in the glyceride-containing feed;
b. separating by phase separation said intermediate to provide a heavier glycerin-containing layer and an intermediate lighter alkyl ester-containing layer, wherein a portion of the water and a portion of the base catalyst are contained in each of the heavier and lighter layer; and
c. contacting the intermediate and lower alkanol wherein the molar ratio of alkanol to glyceride in the intermediate is at least about 3.15:1 to 15:1 under transesterification conditions comprising the presence of a base catalyst for a time sufficient to convert at least 98 mass percent of the glycerides in the glyceride-containing feed and provide a crude biodiesel product.

In some instances, the crude biodiesel product is substantially single phase. Preferably step (c) is conducted in a plug flow reactor.

### BRIEF DESCRIPTION OF THE DRAWINGS

FIG. 1 is a schematic depiction of a biodiesel facility using the processes of this invention.

FIG. 2 is a schematic depiction of another biodiesel facility using the processes of this invention.

### DETAILED DISCUSSION

The following discussion is in reference to the facility depicted in the Figures. The Figures are not intended to be in limitation of this invention.

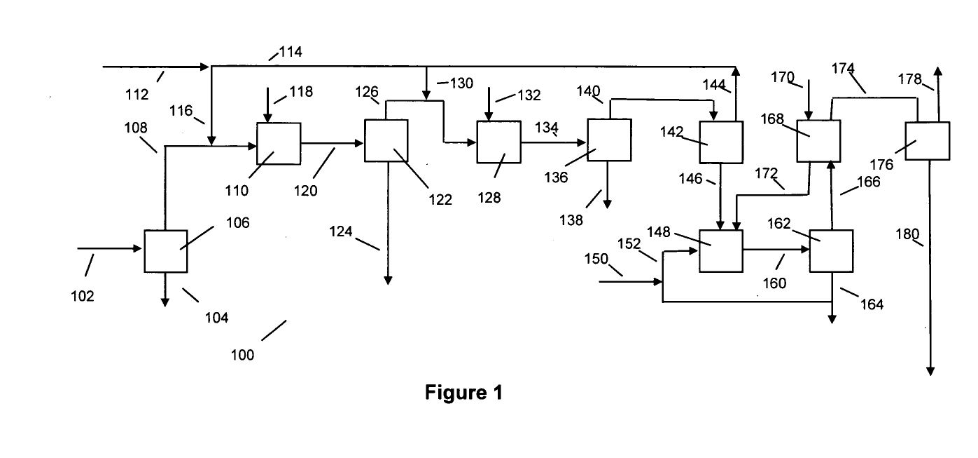

With respect to FIG. 1, biodiesel manufacturing facility 100 uses a suitable raw material feed provided via line 102. The feed may be one or more suitable oils or fats derived from bio sources, especially vegetable oils and animal fats. Examples of fats and oils are rape seed oil, soybean oil, cotton seed oil, safflower seed oil, castor bean oil, olive oil, coconut oil, palm oil, corn oil, canola oil, jatropha oil, rice bran oil, tobacco seed oil, fats and oils from animals, including from rendering plants and fish oils. The oils and fats may contain free fatty acids falling within a broad range. Generally, the free fatty acid in the raw material feed is less than about 60, and unless pretreatment occurs to remove free fatty acids, preferably less than about 10, mass percent (dry basis). The balance of the fats and oils is largely fatty acid triglycerides. The unsaturation of the free fatty acids and triglycerides may also vary over a wide range. Typically, some degree of unsaturation is preferred to reduce the propensity of the biodiesel to gel at cold temperatures.

As shown, the raw material feed in line 102 is passed to pretreatment unit 106 which may effect one or more unit operations to enhance the feed as a transesterification feedstock such as drying, free fatty acid removal, filtration to remove particulates, and the like. Line 104 shows a discharge of rejected material from such unit operations. Reference is made to co-pending PCT patent application (Atty Docket GEIN-102-PCT), filed on even date herewith, and hereby incorporated by reference in its entirety, for processes for removing fatty acids.

A glyceride-containing feed is passed from unit operations 106 via line 108 to reactor 110 for transesterification. The transesterification is base catalyzed with a lower alkanol, preferably methanol, ethanol or isopropanol. Higher alkanols can be used. Methanol is the most preferred alkanol not only due to its availability but also because of its ease of recovery by vapor fractionation. For purposes of the following discussion, methanol will be the alkanol.

As shown, methanol is supplied via line 112 to methanol header 114. Line 116 supplies methanol to reactor 110. Although line 116 is depicted as introducing methanol into line 108, it is also contemplated that methanol can be added directly to reactor 110. Generally methanol is supplied only in a slight excess above that required to effect the sought degree of transesterification in reactor 110. More methanol can be supplied but it may be lost from the facility. Preferably, the amount of methanol is from about 101 to 500, more preferably, from about 105 to 200, mass percent of that required for the sought degree of transesterification in reactor 110. In the facility depicted, two reactors are used. One reactor may be used, but since the reaction is equilibrium limited, most often at least two reactors are used. Often, where more than one reactor is used, at least about 60, preferably between about 70 and 96, percent of the glycerides in the feed are reacted in the first reactor.

The base catalyst is shown as being introduced via line 118 to reactor 110. Preferably, the amount of catalyst is from about 101 to 200, more preferably, from about 101 to 150, mass percent of that required for the sought degree of transesterification in reactor 110. The amount of catalyst used will reflect the amount of base that will react with free fatty acids to form soaps in the transesterification. Free fatty acids may be present in the feed to the reactor as well as be formed as a side product during the transesterification reaction. The base catalyst may be an alkali or alkaline earth metal hydroxide or alkali or alkaline earth metal alkoxide, especially an alkoxide corresponding to the lower alkanol reactant. The preferred alkali metals are sodium and potassium. When the base is added as a hydroxide, it may react with the lower alkanol to form an alkoxide with the generation of water. The exact form of the catalyst is not critical to the understanding and practice of this invention.

The transesterification in reactor 110 is often at a temperature between about 30° C. and 220° C., preferably between about 30° C. and 80° C. The pressure is typically in the range of between about 90 to 500 kPa (absolute) although higher and lower pressures can be used. The reactor is typically batch, semi-batch, plug flow or continuous flow tank with some agitation or mixing, e.g., mechanically stirred, ultrasonic, static mixer, e.g., a packed bed, baffles, orifices, venturi nozzles, tortuous flow path, or other impingement structure. The residence time will depend upon the desired degree of conversion, the ratio of methanol to glyceride, reaction temperature, the degree of agitation and the like, and is often in the range of about 0.1 to 20, say, 0.2 to 10, hours.

The partially transesterified effluent for reactor 110 is passed via line 120 to phase separator 122. Phase separator 122 may be of any suitable design and provides a glycerin-containing bottoms stream passed via line 124. The material in line 124 can be subjected to suitable unit operations to recover components thereof. This stream also contains a portion of the soaps, if any, made in reactor 110 and a portion of the catalyst. The lighter phase contains alkyl esters and unreacted glycerides and is passed via line 126 to second transesterification reactor 128.

Reactor 128 may be of any suitable design and may be similar to or different than reactor 110. As shown, additional methanol is provided via line 130 from methanol header 114 and additional catalyst is provided via line 132. Preferably the transesterification conditions in reactor 128 are sufficient to react at least about 90, more preferably at least about 95, and sometimes at least about 97 to 99.9, mass percent of the glycerides in the feed to reactor 110. The transesterification in reactor 128 is typically operated under conditions within the parameters set forth for reactor 110 although the conditions may be the same or different. The residence time will depend upon the desired degree of conversion. Typically, it is desired that the conversion be at least about 98, preferably at least about 99, percent complete based upon the conversion of the glycerides in the feed.

The effluent from reactor 128 is passed via line 134 to phase separator 136 which may be of any suitable design and may be the same as or different from the design of separator 122. A heavier, glycerine-containing phase is withdrawn via line 138. This stream contains some catalyst and methanol. A lighter phase containing crude biodiesel is withdrawn from separator 136 via line 140. The lighter phase also contains catalyst and methanol.

The crude is then passed without catalyst neutralization to methanol separator 142. Methanol separator 142 effects a fast, vapor fractionation of the lower alkanol from the crude biodiesel and may be of any convenient design including a stripper, wiped film evaporator, falling film evaporator, and the like.

As stated above, a falling film evaporator is the preferred apparatus for effecting the vapor fractionation. The tubes of the falling film evaporator may be circular in cross section or any other convenient cross-sectional shape, and the tubes may have a constant cross-sectional configuration over their length or may be tapered or otherwise change in cross-sectional configuration.

Often the vapor fractionation recovers at least about 70, preferably at least about 90, mass percent of the lower alkanol contained in the crude biodiesel. Any residual alkanol is substantially removed in any subsequent water washing of the crude biodiesel. Advantageously, the amount of alkanol contained in the spent water from the washing may be at a sufficiently low concentration that the water can be disposed without further treatment. However, from a process efficiency standpoint, methanol can be recovered from the spent wash water for recycle to the transesterification reactors.

The lower boiling fraction containing the lower alkanol will contain a portion of any water contained in the crude biodiesel. Since the transesterification is conducted with little water being present, and a portion of the water is removed with the glycerin, the concentration of water in this fraction can be sufficiently low that the lower boiling fraction comprising lower alkanol can be recycled to the transesterification reactors. This lower boiling fraction often contains less than about 0.5, and more preferably less than about 0.3, mass percent water. The methanol-containing fraction is removed from separator 142 via line 144 and may be exhausted from the facility as a waste stream, e.g., for burning or other suitable disposal, or can be added to the methanol header 114. The bottoms stream from methanol separator 142 is contacted with an aqueous acid solution to neutralize the catalyst and any soaps present.

As shown, the bottoms stream is subjected to a strong acid treatment to recover free fatty acids. Often, if only base catalyst neutralization is sought, a much weaker and smaller volume acid solution can be used.

The bottoms stream is passed via line 146 to mixer 148. Into mixer 148 is passed a strong acid aqueous solution via line 152. Mixer 148 may be an in-line mixer or a separate vessel. Mixer 148 should provide sufficient mixing and residence time that essentially all of the soaps are converted to free fatty acids. Often the temperature during the mixing is in the range of about 40° C. to 100° C., and for a residence time of between about 0.01 to 4, preferably 0.02 and 1, hours.

In accordance with the processes of this invention, the strong acid aqueous solution introduced via line 152 has a pH sufficient to convert the soaps to free fatty acids. Often the pH is less than about 6, and more preferably less than about 5, say, between about 2 and 5. The acid may be any suitable acid to achieve the sought pH such as hydrochloric acid, sulfuric acid, sulfonic acid, phosphoric acid, perchloric acid and nitric acid. Sulfuric acid is preferred due to cost and availability.

The effluent from mixer 148 is passed via line 160 to phase separator 162. Phase separator 162 may be of any suitable design. A lower aqueous phase is withdrawn via line 164. A portion of this aqueous phase is purged and the remaining portion is recycled via line 152 to mixer 148. Make-up acid is provided via line 150 to line 152.

The lighter phase which contains crude biodiesel and free fatty acid is withdrawn via line 166 and is passed to water wash column 168. Fresh water enters column 168 via line 170 and serves to remove residual acid, methanol and salts from the crude biodiesel. Water wash column 168 may be of any suitable design. Normally the column is operated at a temperature between about 20° C. and 80° C. or 100° C., preferably between about 35° C. and 75° C.

Instead of a wash column, the water washing of the crude biodiesel may be effected through the use of one or more contact vessels each followed by a decanter to separate the oil phase from the water-containing phase. See, for instance, copending PCT patent application (GEIN-102-PCT), filed on even date herewith.

In a preferred embodiment, the spent water from wash column 168 is passed via line 172 to mixer 148 or combined with the aqueous solution in line 152. Most preferably, the water provided via line 170 is in an amount to replace the volume of purge from line 164 to maintain steady state conditions. Often the purge from line 164 is less than 20, preferably between about 5 and 15, volume percent of the lower aqueous phase withdrawn from separator 162.

A washed biodiesel stream is withdrawn from washing column 168 via line 174 and is passed to drier 176 to remove water and residual methanol which exhaust via line 178. Drier 176 may be of any suitable design such as stripper, wiped film evaporator, falling film evaporator, and solid sorbent. Generally the temperature of drying is between about 80° C. and 220° C., say, about 100° C. and 180° C. An inert gas such as nitrogen can be introduced to enhance the water removal. The dried biodiesel is withdrawn as product via line 180. The biodiesel product contains no more than 0.58, and more preferably less than about 0.25, mass percent.

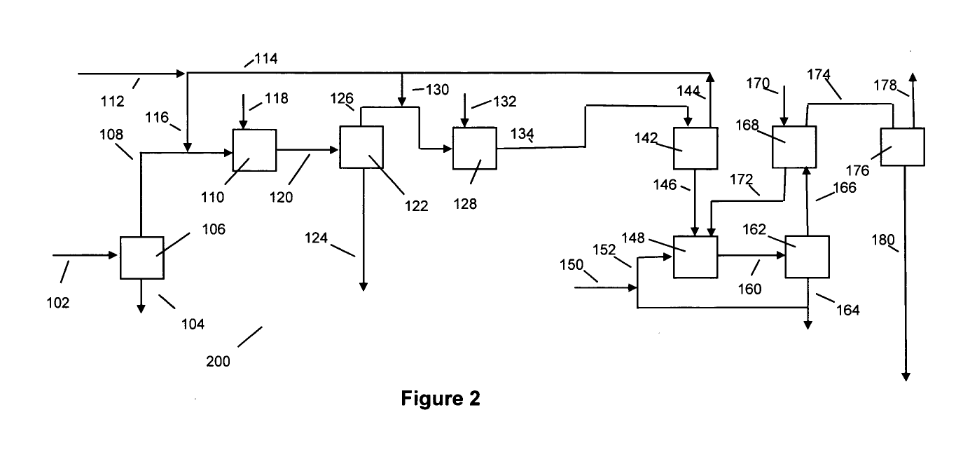

With reference to FIG. 2, the biodiesel manufacturing facility 200 does not include a phase separation unit operation following reactor 128. For purposes of this figure, all similar components are marked with the same identification number and the above descriptions are incorporated herein for such components.

In reactor 110, the conversion of the glycerides in the feed is at least about 90, preferably 92 to 96 or 98, percent. Thus the lighter phase from phase separator 122 contains little glyceride. In reactor 128 the reaction proceeds quickly to completion by the addition of additional methanol. Especially with the higher conversions, the effluent from reactor 128 may be a single phase. The effluent is shown as being directed to falling film evaporator 142 for recovery of methanol.

It is claimed:

1. A process for recovering lower alkanol from a crude biodiesel containing alkyl esters of fatty acids, lower alkanol, and a catalytically effective amount of base catalyst, comprising subjecting the crude biodiesel that contains less than about 0.5 mass percent water to fast, vapor fractionation conditions to provide a lower boiling fraction containing lower alkanol.

2. The process of claim 2 wherein the lower alkanol is at least one of methanol, ethanol and isopropanol.

3. The process of claim 1 wherein the lower alkanol is methanol and the vapor fractionation conditions comprise a maximum temperature of less than about 120° C.

4. The process of claim 3 wherein the vapor fractionation is effected by falling film evaporation.

5. The process of claim 1 wherein the vapor fractionation is effected by falling film evaporation.

6. A process for making biodiesel comprising:
a. contacting a glyceride-containing feed and lower alkanol under transesterification conditions comprising the presence of a base catalyst, wherein the molar ratio of lower alkanol to glyceride is at least about 3.15:1 to provide a crude biodiesel containing alkyl esters of fatty acids, glycerin, lower alkanol, base catalyst, and less than about 0.1 mass percent water, said contacting being for a time sufficient to convert at least about 90 mass percent of the glycerides in the glyceride-containing feed;
b. separating by phase separation said crude biodiesel to provide a heavier glycerin-containing layer and a lighter alkyl ester-containing layer, wherein a portion of the water and a portion of the base catalyst are contained in each of the heavier and lighter layer;
c. subjecting the lighter layer while it contains a catalytically effective amount of base catalyst to vapor fractionation conditions to provide a lower boiling fraction containing lower alkanol and a higher boiling fraction containing alkyl esters and base catalyst;
d. recycling at least a portion of the lower boiling fraction to step (a) as a portion of the lower alkanol; and
e. contacting the higher boiling fraction with an aqueous acid solution in an amount sufficient to at least neutralize the base catalyst.

7. The process of claim 6 wherein step (a) comprises using at least two sequential reaction zones with an intermediate phase separation to remove a heavier, glycerin-containing layer.

8. The process of claim 7 wherein the lower boiling fraction of step (c) is recycled per step (d) without separation of water.

9. The process of claim 8 wherein the lower alkanol is methanol.

10. The process of claim 9 wherein the vapor fractionation is effected by falling film evaporation.

11. An apparatus for conducting the process of claim 9.

12. The apparatus of claim 11 in which a falling film evaporator is used to effect step (c).

13. The apparatus of claim 12 in which the falling film evaporator has tubes of an average diameter of between about 2 and 10 centimeters and a length of at least one meter.

14. A process for making biodiesel comprising:
a. contacting a glyceride-containing feed and lower alkanol under transesterification conditions comprising the presence of a base catalyst, wherein the molar ratio of lower alkanol to glyceride is at least about 3.15:1 to provide an intermediate containing alkyl esters of fatty acids, glycerin, lower alkanol, base catalyst, and less than about 0.1 mass percent water, said contacting being for a time sufficient to convert at least about 90 mass percent of the glycerides in the glyceride-containing feed;
b. separating by phase separation said intermediate to provide a heavier glycerin-containing layer and an intermediate lighter alkyl ester-containing layer, wherein a portion of the water and a portion of the base catalyst are contained in each of the heavier and lighter layer; and
c. contacting the intermediate and lower alkanol wherein the molar ratio of alkanol to glyceride in the intermediate is at least about 3.15:1 under transesterification conditions comprising the presence of a base catalyst for a time sufficient to convert at least 98 mass percent of the glycerides in the glyceride-containing feed and provide a crude biodiesel product.

15. The process of claim 14 wherein the crude biodiesel product is subjected, while it contains a catalytically effective amount of base catalyst, to vapor fractionation conditions to provide a lower boiling fraction containing lower alkanol and a higher boiling fraction containing alkyl esters and base catalyst.

16. The process of claim 14 wherein the crude biodiesel product is substantially single phase.

17. The process of claim 14 wherein step (c) is conducted in a plug flow reactor.

18. The process of claim 14 wherein the lower alkanol is methanol.
